(19) United States
(12) Patent Application Publication
(10) Pub. No.: US 2026/0166330 A1
(43) Pub. Date: Jun. 18, 2026

(54) STIMULATION SIGNAL CONTROL SYSTEM AND METHOD

(71) Applicant: INDUSTRIAL TECHNOLOGY RESEARCH INSTITUTE, Hsinchu (TW)

(72) Inventors: Chun-Hung LIEN, Taipei City (TW); Ting-Shen LIU, Taoyuan City (TW)

(73) Assignee: INDUSTRIAL TECHNOLOGY RESEARCH INSTITUTE, Hsinchu (TW)

(21) Appl. No.: 19/242,421

(22) Filed: Jun. 18, 2025

(30) Foreign Application Priority Data
Dec. 16, 2024 (TW) 113148980

(51) Int. Cl. A61N 5/06; G16H 20/40

(52) U.S. Cl. CPC A61N 5/06; G16H 20/40; A61N 2005/0626; A61N 2005/0632

(57) ABSTRACT

A stimulation signal control system and method. The stimulation signal control method is performed by a control device and comprises: controlling optical stimulation elements to output a first stimulation signal based on a first optical stimulation parameter set among candidate optical stimulation parameter sets, wherein each of the candidate optical stimulation parameter sets includes a flicker value, a brightness value, and a duty cycle of each of the optical stimulation elements; obtaining a feedback signal corresponding to the first stimulation signal; determining whether the feedback signal corresponds to a to-be-optimized result; selecting a second optical stimulation parameter set adjacent to the first optical stimulation parameter set from the candidate optical stimulation parameter sets based on the feedback signal when the feedback signal corresponds to a to-be-optimized result; and controlling the optical stimulation elements to output a second stimulation signal based on the second optical stimulation parameter set.

### CROSS-REFERENCE TO RELATED APPLICATIONS

This non-provisional application claims priority under 35 U.S.C. § 119 (a) on Patent Application No(s). 113148980 filed in Republic of China (Taiwan) on Dec. 16, 2024, the entire contents of which are hereby incorporated by reference.

### BACKGROUND

### 1. Technical Field

The present disclosure relates to a stimulation signal control system and method.

### 2. Related Art

The problem of population aging is becoming increasingly serious, and the care for dementia has also led to significant economic expenditures. In order to delay or even treat dementia, various preventive and therapeutic methods have been developed. For example, current research has shown that inducing the brain to generate brainwaves in specific frequency bands can improve the cognitive function of users.

### SUMMARY

According to an embodiment of the stimulation signal control method of the present disclosure, is performed by a control device and comprises: controlling a plurality of optical stimulation elements to output a first stimulation signal based on a first optical stimulation parameter set among a plurality of candidate optical stimulation parameter sets, wherein each of the plurality of candidate optical stimulation parameter sets includes a flicker value, a brightness value, and a duty cycle of each of the plurality of optical stimulation elements; obtaining a feedback signal corresponding to the first stimulation signal; determining whether the feedback signal corresponds to a to-be-optimized result; selecting a second optical stimulation parameter set adjacent to the first optical stimulation parameter set from the plurality of candidate optical stimulation parameter sets based on the feedback signal when the feedback signal corresponds to a to-be-optimized result; and controlling the plurality of optical stimulation elements to output a second stimulation signal based on the second optical stimulation parameter set.

A stimulation signal control system, comprising: a plurality of optical stimulation elements, a storage device, an input device and a control device. The storage device is configured to store a plurality of candidate optical stimulation parameter sets, wherein each of the plurality of candidate optical stimulation parameter sets includes a flicker value, a brightness value, and a duty cycle of each of the plurality of optical stimulation elements. The input device is configured to obtain a feedback signal. The control device is connected to the plurality of optical stimulation elements, the storage device, and the input device, wherein the control device is configured to perform: controlling the plurality of optical stimulation elements to output a first stimulation signal based on a first optical stimulation parameter set among the plurality of candidate optical stimulation parameter sets; obtaining a feedback signal corresponding to the first stimulation signal from the input device; determining whether the feedback signal corresponds to a to-be-optimized result; selecting a second optical stimulation parameter set adjacent to the first optical stimulation parameter set from the plurality of candidate optical stimulation parameter sets based on the feedback signal when the feedback signal corresponds to the to-be-optimized result; and controlling the plurality of optical stimulation elements to output a second stimulation signal based on the second optical stimulation parameter set.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram of a stimulation signal control system according to an embodiment of the present disclosure;

FIG. 2 is a flowchart of the stimulation signal control method according to an embodiment of the present disclosure; and

FIG. 3 illustrates a stimulation signal output by each optical stimulation element and a first stimulation signal output by the optical stimulation elements according to an embodiment of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

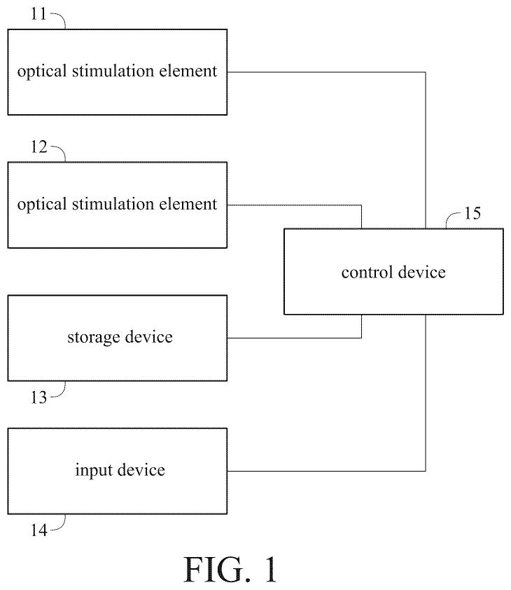

Please refer to FIG. 1, wherein FIG. 1 is a block diagram of a stimulation signal control system according to an embodiment of the present disclosure. The stimulation signal control system 1 may be implemented as an independent device, such as a head-mounted device, an upright device, or the like. As shown in FIG. 1, the stimulation signal control system 1 includes a plurality of optical stimulation elements 11 and 12, a storage device 13, an input device 14, and a control device 15. The control device 15 is electrically or communicatively connected to the optical stimulation elements 11 and 12, the storage device 13, and the input device 14.

The optical stimulation elements 11 and 12 may be light emitting diodes configured to emit light under control. The optical stimulation elements 11 and 12 may correspond to the same frequency, such as 40 Hz, but the present disclosure is not limited thereto. Furthermore, the optical stimulation elements 11 and 12 may emit light at the same frequency.

Additionally, the optical stimulation elements 11 and 12 may emit different colors of light to produce metameric light of the same frequency through mixing different colors of light. FIG. 1 exemplarily illustrates two optical stimulation elements. However, the number of optical stimulation elements may also be greater than two.

The storage device 13 may be a non-volatile memory (NVM), such as read-only memory (ROM), flash memory, and/or non-volatile random-access memory (NVRAM), etc. The storage device 13 configured to store a plurality of candidate optical stimulation parameter sets, wherein each of the plurality of candidate optical stimulation parameter sets includes a flicker value, a brightness value, and a duty cycle of each of the plurality of optical stimulation elements.

Table 1 below is an example of the candidate optical stimulation parameter sets. Take the first candidate optical stimulation parameter set as an example, the light intensities of the optical stimulation elements 11 and 12 are 80% and 30%, respectively; the brightness values of the optical stimulation elements 11 and 12 are 110%; and the flicker values of the optical stimulation elements 11 and 12 are 50%. The sum of the light intensities may be regarded as the brightness value of the candidate optical stimulation parameter set, and the difference in light intensities may be regarded as the flicker value of the candidate optical stimulation parameter set. When the sum of the light intensities is higher, the brightness value of the corresponding stimulation signal is higher; and when the difference in light intensities is higher, the flicker value of the corresponding stimulation signal is higher. The candidate optical stimulation parameter sets may be stored in a tabular form in the storage device 13 based on the order of the flicker values and the order of the brightness values. As shown in FIG. 1, the candidate optical stimulation parameter sets are arranged in the order of the flicker values and the brightness values. In particular, arranging the candidate optical stimulation parameter sets in a two-dimensional table format based on flicker values and brightness values facilitates the efficient selection of adjacent parameter sets. This tabular organization simplifies the process of locating neighboring values when adjusting the stimulation signal and contributes to reducing the computational burden on the control device.

TABLE 1

| first candidate optical | | second candidate optical | | third candidate optical | |
|---|---|---|---|---|---|
| stimulation parameter set | | stimulation parameter set | | stimulation parameter set | |
| 80 | 30 | 90 | 20 | 100 | 10 |
| brightness values: 110 | | brightness values: 110 | | brightness values: 110 | |
| flicker values: 50 | | flicker values: 70 | | flicker values: 80 | |
| forth candidate optical | | fifth candidate optical | | six candidate optical | |
| stimulation parameter set | | stimulation parameter set | | stimulation parameter set | |
| 70 | 30 | 80 | 20 | 90 | 10 |
| brightness values: 100 | | brightness values: 100 | | brightness values: 100 | |
| flicker values: 40 | | flicker values: 60 | | flicker values: 80 | |
| seventh candidate optical | | eighth candidate optical | | ninth candidate optical | |
| stimulation parameter set | | stimulation parameter set | | stimulation parameter set | |
| 60 | 30 | 70 | 20 | 80 | 10 |

| | | |
|---|---|---|
| brightness values: 90 | brightness values: 90 | brightness values: 90 |
| flicker values: 30 | flicker values: 50 | flicker values: 70 |

Additionally, at least a portion of the candidate optical stimulation parameter sets may have the same brightness value. Furthermore, take Table 1 as an example, the first candidate optical stimulation parameter set to the third candidate optical stimulation parameter set may have the same brightness value, the fourth candidate optical stimulation parameter set to the sixth candidate optical stimulation parameter set may have the same brightness value, and the seventh candidate optical stimulation parameter set to the ninth candidate optical stimulation parameter set may have the same brightness value.

It should be noted that in the present disclosure, the light intensity is expressed as a percentage of the brightness of the light emitted by of the optical stimulation element relative to the maximum brightness of the optical stimulation element, where the maximum brightness of the optical stimulation element is represented as 100%.

Furthermore, each candidate optical stimulation parameter set further includes a plurality of duty cycle combinations. For example, for each candidate optical stimulation parameter set, the first duty cycle combination includes a duty cycle of 40% for the optical stimulation element 11 and 60% for the optical stimulation element 12; the second duty cycle combination includes a duty cycle of 50% for both the optical stimulation elements 11 and 12; and the third duty cycle combination includes a duty cycle of 60% for the optical stimulation element 11 and 40% for the optical stimulation element 12.

The brightness values, flicker values, duty cycles, and the number of candidate optical stimulation parameters of the above are only examples, and the present disclosure is not limited thereto. In particular, arranging the candidate optical stimulation parameter sets in a two-dimensional table format based on flicker values and brightness values facilitates the efficient selection of adjacent parameter sets. This tabular organization simplifies the process of locating neighboring values when adjusting the stimulation signal and contributes to reducing the computational burden on the control device.

The input device 14 may include one or more of a brightness sensor, one or more physiological sensors and a user interface device (mouse, keyboard, button or touchscreen). The input device 14 is configured to receive a feedback signal of the user's response to the stimulation signal. The control device 15 is configured to control the optical stimulation elements 11 and 12 to output the stimulation signal, and to determine whether to adjust the stimulation parameters of the optical stimulation elements 11 and 12 based on the feedback signal. The control device 15 may include one or more processors, and the processor is, for example, a central processing unit (CPU), a graphics processing unit (GPU), a microcontroller, a programmable logic controller (PLC), or other processors with signal processing capabilities. In addition to the exemplified sensors such as pupil sensors and galvanic skin sensors, the input device may optionally include other physiological sensors capable of reflecting user discomfort. These may include, for example, heart rate sensors, electrodermal activity sensors, or other biosignal detection devices that provide objective feedback on the user's physiological responses to optical stimulation.

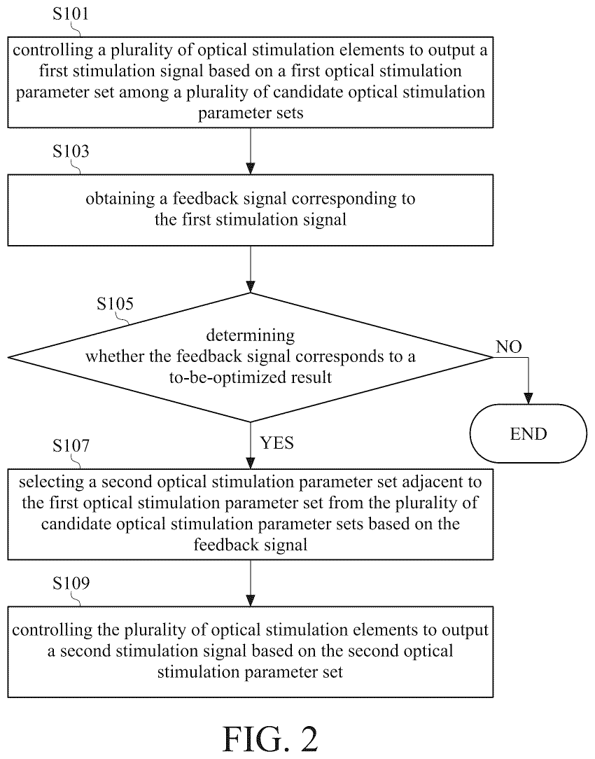

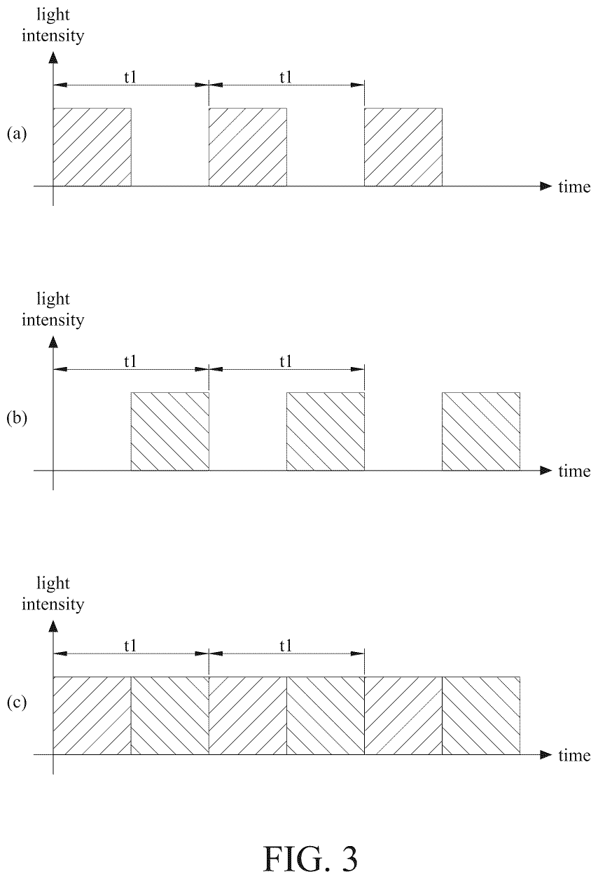

Please refer to FIG. 1, FIG. 2, and FIG. 3 together. FIG. 2 is a flowchart of the stimulation signal control method according to an embodiment of the present disclosure. FIG. 3 illustrates the stimulation signal output by each optical stimulation element and the first stimulation signal output by the plurality of optical stimulation elements according to an embodiment of the present disclosure. As shown in FIG. 2, the stimulation signal control method includes: step S101: controlling a plurality of optical stimulation elements to output a first stimulation signal based on a first optical stimulation parameter set among a plurality of candidate optical stimulation parameter sets; step S103: obtaining a feedback signal corresponding to the first stimulation signal; step S105: determining whether the feedback signal corresponds to a to-be-optimized result; when the determination result of step S105 is “yes”, performing step S107: selecting a second optical stimulation parameter set adjacent to the first optical stimulation parameter set from the plurality of candidate optical stimulation parameter sets based on the feedback signal; step 109: controlling the plurality of optical stimulation elements to output a second stimulation signal based on the second optical stimulation parameter set; and when the determination result of step S105 is “no”, the method is ended.

In step S101, the control device 15 selects one of the candidate optical stimulation parameter sets as the first optical stimulation parameter set. For example, in Table 1, the control device 15 may select the fifth candidate optical stimulation parameter set, which has intermediate flicker value and intermediate brightness value, as the first optical stimulation parameter set from the plurality of candidate optical stimulation parameter sets.

The control device 15 controls the optical stimulation element 11 to output light with an intensity of 80% and the optical stimulation element 12 to output light with an intensity of 20%, based on the two light intensities in the first optical stimulation parameter set and the duty cycle of the first optical stimulation parameter set. The control device 15 may control the optical stimulation elements 11 and 12 to emit light simultaneously, so that the light emitted by the optical stimulation elements 11 and 12 is combined to form the first stimulation signal. For example, part (a) of FIG. 3 presents the stimulation signal output from the optical stimulation element 11, part (b) of FIG. 3 presents the stimulation signal output from the optical stimulation element 12, and part (c) of FIG. 3 presents the first stimulation signal after the stimulation signal output by the optical stimulation element 11 and the stimulation signal output by the optical stimulation element 12 are combined. The control device 15 controls the optical stimulation element 11 to first output the stimulation signal, and controls the optical stimulation element 11 to output a stimulation signal with the same light intensity after each time interval t1, as shown in part (a) of FIG. 3; and the control device 15 controls the optical stimulation element 12 to start outputting a stimulation signal with the same light intensity every time interval t1 after half of the time interval t1 has passed, as shown in part (b) of FIG. 3. In other words, as shown in part (c) of FIG. 3, the control device 15 may control the optical stimulation elements 11 and 12 to alternately and continuously output stimulation signals to form the first stimulation signal. FIG. 3 is explained using a duty cycle of 50% as an example, but the present disclosure is not limited thereto.

In step S103, the control device 15 receives a feedback signal in response to the first stimulation signal from the input device 14. For example, step S103 may include the input device 14 receiving a user command as the feedback signal when the input device 14 is a user interface device; step S103 may include using the brightness sensor to measure and obtain the ambient light brightness as the feedback signal when the input device 14 is a brightness sensor; step S103 may include using the pupil sensor to measure and obtain the pupil size as the feedback signal when the input device 14 is a pupil sensor; and step S103 may include using the galvanic skin sensor to measure and obtain the galvanic skin signal as the feedback signal when the input device 14 is a galvanic skin sensor. In addition to the exemplified sensors such as pupil sensors and galvanic skin sensors, the input device may optionally include other physiological sensors capable of reflecting user discomfort. These may include, for example, heart rate sensors, electrodermal activity sensors, or other biosignal detection devices that provide objective feedback on the user's physiological responses to optical stimulation.

In step S105, the control device 15 determines whether the feedback signal corresponds to the to-be-optimized result. For example, the control device 15 determines that the feedback signal corresponds to the to-be-optimized result when the user command indicates that the brightness value of the first light stimulation parameter set is too high or too low, or that the flicker value of the first light stimulation parameter set is too high or too low; the control device 15 determines that the feedback signal corresponds to the to-be-optimized result when the ambient light brightness falls outside of a preset brightness range; the control device 15 determines that the feedback signal corresponds to the to-be-optimized result when the pupil size is smaller than a preset size; and the control device 15 determines that the feedback signal corresponds to the to-be-optimized result when the amplitude of the galvanic skin signal is greater than a preset amplitude. The preset brightness range, the preset size, and the preset amplitude can be preset according to user requirements. Furthermore, as mentioned above, the input device 14 may include more than one of the devices of the user interface device, the brightness sensor, the pupil sensor, and the galvanic skin sensor at the same time, and the control device 15 may perform step S107 when determining that the feedback signal from at least one of the plurality of devices corresponds to the to-be-optimized result.

In step 107, the control device 15 selects the second optical stimulation parameter set which is adjacent to the first optical stimulation parameter sets from the plurality of candidate optical stimulation parameter sets. Take Table 1 as an example, the control device 15 may select the eighth candidate optical stimulation parameter set with a lower brightness value as the second optical stimulation parameter set when the user command indicates that the brightness value of the first optical stimulation parameter set is too high; the control device 15 may select the eighth candidate optical stimulation parameter set with a lower brightness value as the second optical stimulation parameter set when the ambient light brightness is lower than a lower limit of the preset brightness range; the control device 15 may select the second candidate optical stimulation parameter set with a higher brightness value as the second optical stimulation parameter set when the ambient light brightness is higher than an upper limit of the preset brightness range; the control device 15 may select the eighth candidate optical stimulation parameter set with a lower brightness value as the second optical stimulation parameter set, or select the fourth candidate optical stimulation parameter set with a lower flicker value as the second optical stimulation parameter set when the pupil size is smaller than the preset size; the control device 15 may select the eighth candidate optical stimulation parameter set with a lower brightness value as the second optical stimulation parameter set, or select the fourth candidate optical stimulation parameter set with a lower flicker value as the second optical stimulation parameter set when the amplitude of the galvanic skin signal is greater than the preset amplitude. Alternatively, the control device 15 may also select an optical stimulation parameter set with the same brightness value and same flicker value as the fifth optical stimulation parameter set but with a different duty cycle as the second optical stimulation parameter set.

Furthermore, in the embodiment in which the input device 14 includes the plurality of devices including the user interface device, the brightness sensor, the pupil sensor, and the galvanic skin sensor, the control device 15 may use the user command as the primary basis and select the candidate optical stimulation parameter set with a lower brightness value as the second optical stimulation parameter set when the feedback signals from the plurality of devices correspond to different results (for example, the user command indicates that the brightness value of the first optical stimulation parameter set is too high, and the pupil size is not smaller than the preset size.)

In step 109, the control device 15 controls the optical stimulation elements 11 and 12 to output the second stimulation signal based on the second optical stimulation parameter set. The implementation of step S109 can be the same as that of step S101 and descriptions thereof are not be repeated herein.

The control device 15 may end the method or store the first optical stimulation parameter set into the storage device 13 as the default optical stimulation parameter set when the determination result of step S105 is “no”, the present disclosure is not limited thereto.

Furthermore, the control device 15 may perform step S103 again to obtain the feedback signal corresponding to the second stimulation signal after step S109 is performed.

In view of the above description, the stimulation signal control system and method according to one or more embodiments of the present disclosure, allows user to adjust sensory stimulation signals appropriately without the need to visit medical institutions by using the feedback signal as the basis for adjusting stimulation parameters, which enables brain stimulation to be easily integrated into daily usage scenarios and rapidly alleviates user's discomfort. Furthermore, by storing the candidate optical stimulation parameter sets in a tabular form and arranging the candidate optical stimulation parameter sets in order, the control device does not need to recalculate the appropriate optical stimulation parameter set, thereby reducing the computational load on the control device.

It will be apparent to those skilled in the art that various modifications and variations may be made to the disclosed embodiments. It is intended that the specification and examples be considered as exemplars only, with a true scope of the disclosure being indicated by the following claims and their equivalents.

What is claimed is:

1. A stimulation signal control method, performed by a control device and comprising:
controlling a plurality of optical stimulation elements to output a first stimulation signal based on a first optical stimulation parameter set among a plurality of candidate optical stimulation parameter sets, wherein each of the plurality of candidate optical stimulation parameter sets includes a flicker value, a brightness value, and a duty cycle of each of the plurality of optical stimulation elements;
obtaining a feedback signal corresponding to the first stimulation signal;
determining whether the feedback signal corresponds to a to-be-optimized result;
selecting a second optical stimulation parameter set adjacent to the first optical stimulation parameter set from the plurality of candidate optical stimulation parameter sets based on the feedback signal when the feedback signal corresponds to a to-be-optimized result; and
controlling the plurality of optical stimulation elements to output a second stimulation signal based on the second optical stimulation parameter set.

2. The stimulation signal control method according to claim 1, wherein the plurality of optical stimulation elements are configured to emit light of different colors of light to form the first stimulation signal.

3. The stimulation signal control method according to claim 1, wherein the plurality of candidate optical stimulation parameter sets have the same brightness value.

4. The stimulation signal control method according to claim 1, wherein the plurality of candidate optical stimulation parameter sets are stored in a tabular form based on an order of the flicker value and an order of the brightness value.

5. The stimulation signal control method according to claim 1, wherein obtaining the feedback signal corresponding to the first stimulation signal comprises:
obtaining ambient light brightness from a brightness sensor as the feedback signal,
wherein the feedback signal corresponds to the to-be-optimized result when the ambient light brightness falls outside of a preset brightness range.

6. The stimulation signal control method according to claim 1, wherein obtaining the feedback signal corresponding to the first stimulation signal comprises:
obtaining a pupil size from a pupil sensor as the feedback signal,
wherein the feedback signal corresponds to the to-be-optimized result when the pupil size is smaller than a preset size.

7. The stimulation signal control method according to claim 1, wherein obtaining the feedback signal corresponding to the first stimulation signal comprises:
obtaining a galvanic skin signal from a skin electrical sensor as the feedback signal,
wherein the feedback signal corresponds to the to-be-optimized result when an amplitude of the galvanic skin signal is greater than a preset amplitude.

8. The stimulation signal control method according to claim 1, wherein the plurality of optical stimulation elements correspond to a same frequency.

9. A stimulation signal control system, comprising:
a plurality of optical stimulation elements;
a storage device configured to store a plurality of candidate optical stimulation parameter sets, wherein each of the plurality of candidate optical stimulation parameter sets includes a flicker value, a brightness value, and a duty cycle of each of the plurality of optical stimulation elements;
an input device configured to obtain a feedback signal; and
a control device, connected to the plurality of optical stimulation elements, the storage device, and the input device, wherein the control device is configured to preform:
controlling the plurality of optical stimulation elements to output a first stimulation signal based on a first optical stimulation parameter set among the plurality of candidate optical stimulation parameter sets;
obtaining the feedback signal corresponding to the first stimulation signal from the input device;
determining whether the feedback signal corresponds to a to-be-optimized result;
selecting a second optical stimulation parameter set adjacent to the first optical stimulation parameter set from the plurality of candidate optical stimulation parameter sets based on the feedback signal when the feedback signal corresponds to the to-be-optimized result; and
controlling the plurality of optical stimulation elements to output a second stimulation signal based on the second optical stimulation parameter set.

10. The stimulation signal control system according to claim 9, wherein the plurality of optical stimulation elements are configured to emit different colors of light to form the first stimulation signal.

11. The stimulation signal control system according to claim 9, wherein the plurality of candidate optical stimulation parameter sets have the same brightness value.

12. The stimulation signal control system according to claim 9, wherein the plurality of candidate optical stimulation parameter sets are stored in a tabular form based on an order of the flicker value and an order of the brightness value.

13. The stimulation signal control system according to claim 9, wherein the input device is a brightness sensor, and the control device obtains the ambient light brightness from the brightness sensor as the feedback signal, wherein the feedback signal corresponds to the to-be-optimized result when the ambient light brightness falls outside of a preset brightness range.

14. The stimulation signal control system according to claim 9, wherein the input device is a pupil sensor, and the control device obtains a pupil size from the pupil sensor as the feedback signal, wherein the feedback signal corresponds to the to-be-optimized result when the pupil size is smaller than a preset size.

15. The stimulation signal control system according to claim 9, wherein the input device is a galvanic skin sensor, and the control device obtains a galvanic skin signal from the galvanic skin sensor as the feedback signal, wherein the feedback signal corresponds to the to-be-optimized result when an amplitude of the galvanic skin signal greater than a preset amplitude.

16. The stimulation signal control system according to claim 9, wherein the plurality of optical stimulation elements correspond to a same frequency.

17. The stimulation signal control system according to claim 9, wherein the input device is selected from a group consisting of a pupil sensor, a galvanic skin sensor, or another physiological sensor configured to detect user discomfort.
